(19) United States
(12) Patent Application Publication
(10) Pub. No.: US 2019/0360959 A1
(43) Pub. Date: Nov. 28, 2019

(54) A SENSOR

(71) Applicant: AGENCY FOR SCIENCE, TECHNOLOGY AND RESEARCH, Singapore (SG)

(72) Inventors: Jackie Y. YING, Singapore (SG); Jun Hui SOH, Singapore (SG)

(21) Appl. No.: 16/484,821

(22) PCT Filed: Feb. 9, 2018
(86) PCT No.: PCT/SG2018/050060

§ 371 Date: Aug. 8, 2019

(30) Foreign Application Priority Data
Feb. 9, 2017 (SG) 10201701062Q

(51) Int. Cl. G01N 27/333; A61B 5/00; A61F 13/42; G01N 33/493; G01N 27/327

(52) U.S. Cl. CPC G01N 27/333; A61B 5/002; G01N 27/327; A61B 5/6808; G01N 33/493; A61F 13/42

(57) ABSTRACT

The present invention relates to a sensor for detecting one or more target analytes, the sensor comprising: at least one polymeric sensing element capable of selectively and reversibly binding to a target analyte; at least one working electrode having the polymeric sensing element disposed thereon; at least one reference electrode that is electrically communicated with said working electrode; and means for measuring a change in an electrical property across said working electrode and said reference electrode. In particular, the target analyte is Na+, urea or creatinine. Also disclosed is a multi-layered sensor, comprising at least one working electrode layer and at least one reference electrode layer, said working electrode layer and said reference electrode layer being separated by at least one electrically insulating layer.

### TECHNICAL FIELD

The present invention generally relates to a sensor and methods of using the same in real-time health-screening, monitoring and diagnostic applications.

### BACKGROUND ART

Urine is a useful specimen for diagnostic and health screening as it can be collected in large volumes non-invasively. Furthermore, the processing and storage of urine is significantly easier when compared to tissue biopsies and other body fluids, such as whole blood, serum/plasma, and saliva. Urine can be used to detect infection (bacterial and viral), inflammation, cancers (e.g. bladder cancer), and drugs abuse. In particular, urine can be used as a vital early indicator for urinary tract infection (UTI), kidney disease and diabetes, which are asymptomatic in the early stages and pose risks of severe damage if undetected and left untreated. For instance, an abnormally high concentration of urinary urea and creatinine may be prognostic towards renal failure, which is a global health issue. In addition, the concentration of specific electrolytes in urine, such as sodium (Na+), can be used to monitor dehydration, which may have severe consequences, such as lethargy, confusion, seizures and fainting, especially for the elderly.

Urinalysis is commonly conducted using a dipstick. Proper functioning of the liver and kidneys, as well as the presence of UTI can be determined through colorimetric-based chemical reactions on a urine dipstick. Although results from urine dipsticks can be easily and conveniently read, sensitivity and selectivity issues are a concern. For more accurate and reliable urinalysis, clinical analysis in laboratories can be performed on collected urine samples. However, collecting samples from patients poses inconvenience, especially if repeated sampling has to be conducted throughout the day. Also, clinical analyses usually have a long turnaround time, and are unsuitable for use in the field and at the point-of-care (POC).

Wearable electrochemical sensors that can be worn and integrated with an individual's daily routine would be vital to enabling personalized medicine by continuous, real-time monitoring of the individual's health status. Currently, diaper sensor technologies are limited to the detection of urine and/or feces by detecting wetness, humidity, and temperature. These devices are unable to determine the wearer's health status at the molecular levels and are also unable to provide real-time data. Furthermore, these technologies utilize expensive materials, such as humidity sensors, printed circuit boards and light-dependent resistor, for sensing.

Hence, there is a need to develop sensors which are portable, sensitive, and provide reliable urinalysis. It is also desired to develop a sensor that is capable of providing real-time data and monitoring.

### SUMMARY OF INVENTION

According to a first aspect of the present disclosure, there is provided a sensor for detecting one or more target analytes, the sensor comprising: at least one polymeric, sensing element capable of selectively and reversibly binding to a target analyte; at least one working electrode having the polymeric sensing element disposed thereon; at least one reference electrode that is electrically communicated with said working electrode; and means for measuring an electrical property across said working electrode and said reference electrode, wherein a change in the electrical property is indicative of the presence of the target analyte.

Advantageously, the disclosed sensor may be an electrochemical-based sensor that is capable of measuring the levels of important urinary analytes (e.g., Na+, urea and creatinine) directly from human urine for health screening and monitoring purposes.

Advantageously, the disclosed sensors may utilize inexpensive copper as an electrode material and only require facile modification methods to fabricate.

In certain embodiments, the disclosed sensor is provided in a strip form or strip-based structure. Advantageously, its simple, strip-based structure enables the sensors to be easily inserted into apparel worn by a human or animal subject, (e.g., diapers). This in turn allows the sensor to provide real-time data and continuous analysis of urine samples of the subject.

Accordingly, the present disclosure also relates to in vitro diagnostic (IVD) devices comprising the sensors disclosed herein.

### Definitions

The following words and terms used herein shall have the meaning indicated:

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### DETAILED DISCLOSURE OF EMBODIMENTS

Exemplary, non-limiting embodiments of a sensor according to the present disclosure will now be disclosed.

The sensor may comprise at least two or more polymeric sensing elements, which may be discretely and separately disposed on the working electrode. Each polymeric sensing element may be independently configured to detect the same or different target analyte. In one embodiment, the sensor may comprise at least three polymeric sensing elements, each sensing element being located discretely and separately from each other and being configured to detect a different analyte from the other sensing elements.

The sensor may be provided in a strip-like structure, wherein its total thickness is between 85 to 150 μm. The width of the sensor strip may be from 5 to 10 mm wide; whereas the length of the sensor may be from around 450 to 750 mm long.

When in use, the polymeric sensing elements may be concurrently exposed to an external environment, wherein the sensing elements may come into contact with a fluid or liquid potentially containing the target analytes. The liquid may be urine. The external environment may be the interior space of a diaper. The sensor may not be in direct contact with the human body. For instance, the sensor may be substantially enclosed by a semi-permeable membrane permitting the ingress of the target analytes thereof.

The electrodes of the sensor may be composed of any suitable conductive metal substrate. The electrode may also be composed of a material that is substantially chemically inert with respect to the target analytes intended for detection and measurement. In one embodiment, the electrodes are composed of copper.

Each polymeric sensing element may be independently selected from an ion-selective polymer membrane or a molecularly imprinted polymer (MIP) film. The selection of the polymeric sensing element may depend on the specific nature of the analyte to be detected. For instance, where the analyte is a molecule, a MIP film may be selected as the sensing element.

Where the polymeric sensing element is an ion-selective polymer membrane, it may comprise an ionophore dispersed within a polymer matrix, wherein the ionophore is capable of reversibly forming a complex with said target analyte. The polymer matrix may further comprise at least one additive selected to repel non-target molecules or ions, which are not of the same charge as the target analyte, from the ion-selective polymer membrane. The additive may be a lipophilic ion additive, which advantageously ensures that the membrane is only permeable to ions or analytes with the same charge sign as the target analyte. In certain embodiments, the ion-selective polymer membrane may be prepared from a polymer coating composition comprising at least one polymer, a plasticizer, an ionophore and at least one lipophilic ion additive. The coating composition may also comprise one or more organic solvents. The preparation step may comprise casting, spin-coating or dipping. The preparation may also comprise a step of allowing the casted polymer layer to dry. Optionally, the dried polymer may be subjected to a washing step.

The polymeric sensing elements may also be provided in as a multi-layered structure, wherein one or more additional layers are deposited over the polymer membrane layer/MIP film that is disposed directly on the electrode surface. For instance, these additional layers may comprise one or more enzymatic coatings to convert one or more target molecules into one or more ionic species for ready detection by an ion-selective polymer membrane. Such a configuration may advantageously permit the detection of plural target analytes on the same locus of the sensor. In one embodiment, a urease layer may be provided as the additional layer in combination with an ion-selecitve polymer membrane configured to detect ammonium ions.

The polymer may be a polyvinyl chloride (PVC) polymer, which provides structural support and strength to the membrane. The polymer may be substantially inert with respect to the analytes to be detected so as to prevent any chemical reaction between the polymer and the analyte. Other suitable polymers may include silicone rubber, polyacrylate, polyurethane, fluoro-polymers (e.g., Teflon AF2400), and co-polymers and mixtures thereof.

The ionophore may be one that is adapted for reversible binding with a sodium ion. In one embodiment, the ionophore is a 4-tert-butylcalix[4]arene-tetraacetic acid tetraethyl ester (marketed as Sodium Ionophore X™ by Sigma Aldrich). The ionophores may be selected based on the ion intended for detection and reversible binding. For instance, where the target ion is sodium, suitable ionophores may include, but are not limited to, sodium ionophore I (ETH 227, N,N′,N″-Triheptyl-N,N′,N″-trimethyl-4,4′,4″-propylidynetris(3-oxabutyramide)), sodium ionophore II (ETH 157, N,N′-Dibenzyl-N,N′-diphenyl-1,2-phenylenedioxydiacetamide), sodium ionophore III (ETH 2120, N,N,N′,N′-Tetracyclohexyl-1,2-phenylenedioxydiacetamide), sodium ionophore IV (2,3:11,12-Didecalino-16-crown-5,2,6,13,16,19-pentaoxapentacyclo[18.4.4.47,12.01,20.07,12]dotriacontane), sodium ionophore V (ETH _{4120, 4}-Octadecanoyloxymethyl-N, N, N′,N′-tetracyclohexyl-1,2-phenylenedioxydiacetamide), sodium ionophore VI (Bis[(12-crown-4)methyl]dodecylmethylmalonate), sodium ionophore VIII (Bis[(12-crown-4)methyl] 2,2-didodecylmalonate), and combinations thereof.

The ionophore, may be a neutral ion carrier, which contains cavities the size of their respective target analyte ions or molecules. The ionophore may be able to selectively form a reversible complex with these target ions or charged molecules. The ionophore may provide the required selectivity of the ion-selective membrane. Exemplary analytes detectable using an ion-selective polymer membrane may include K⁺, Na⁺, NH₄⁺, Ca²⁺, and/or Mg^{2+.}

In one embodiment, the detection of Na⁺ may be conducted using ion-selective polymer membrane coated on conductive copper tape as the working electrode, coupled with an Ag/AgCl coating on another piece of copper tape as the reference electrode for stable potentiometric measurement in sample solutions. Advantageously, polymer-based ion-selective electrodes are versatile since they are easy to produce, inexpensive, and can be easily miniaturized for portable, on-site measurements.

The lipophilic ion additive may be sodium tetrakis [3,5-bis(trifluoromethyl)phenyl]borate. Other suitable additives for improving the selectivity of the polymer membrane may include, but are not limited to, potassium tetrakis(p-chlorophenyl)borate (KTpCIPB), sodium tetraphenylborate, and mixtures thereof.

In a preferred embodiment. the solvent may be tetrahydrofuran (THF). However other suitable solvents may be used alternatively or in combination with THF. Suitable solvents may include, but are not limited to, toluene, acetone, methyl acetate, ethyl acetate, hexane or mixtures thereof.

The plasticizer may be an ester e.g, a dioctyl sebacate. Other suitable plasticizers may include, but are not limited to, bis(1-butylpentyl) adipate, 2-Nitrophenyl octyl ether, Bis(2-ethylhexyl) phthalate, tris(ethylhexyl) phosphate, Chloroparaffin, and/or mixtures thereof. Advantageously, the plasticizer provides a homogeneous organic phase and enables mobility of membrane constituents

Where the polymeric sensing element is a molecularly imprinted polymer (MIP) film, the MIP film may be prepared by: casting a polymer film from a composition comprising a polymer and a target analyte intended for detection by the MIP film; drying the film; and removing the target analyte from the dried film to generate cavities thereon, wherein the cavities are specifically adapted to receive the target analyte. In particular, the MIP film may be prepared by template polymerization of the polymer in the presence of the target analyte. In one embodiment, the MIP film may be prepared by casting a polymer solution comprising poly(vinyl alcohol-co-ethylene) mixed with an organic solvent, e.g., DMSO (dimethyl sulfoxide) and having urea molecules dissolved therein. The casted film may be allowed to dry and thereafter washed with an appropriate solvent (e.g., ethanol) to remove the urea molecules from the MIP film. In another embodiment, the MIP film may be prepared similarly but with creatinine molecules acting as the template molecule for polymerization.

The target analyte may be selected from one or more of the group consisting of: Na+, urea, and creatinine. In embodiments, the sensor is configured to concurrently and independently detect Na⁺, urea, creatinine or metabolites thereof and to determine the concentrations of these analytes in a urine sample.

The working electrode and reference electrode may be separated by an electrically insulating layer. In other words, while the working electrode and reference electrode may be substantially insulated from each other, both electrodes may be electrically communicated with one or more of a potentiometer, rheostat, or an ohmmeter.

The reference electrode may be coated with a reference electrode coating, e.g., a Ag/AgCl coating.

The means for measuring the potential difference or impedance may further comprise at least one transmitter capable of relaying the measured electrical property or changes to electrical property as electrical signals to an external computer for storage, analysis, and output.

The electrical property being measured may be selected from potential difference, impedance or resistivity. The total potential difference, or electromotive force (EMF), may be described as the sum of a constant potential and the membrane potential. When ionophores form complexes with target ions at the phase boundary between the polymer membrane and a sample solution, ion exchange across the phase boundary causes a change in potential difference. This change in potential difference may be detectable by a voltage change.

On the other hand, where the target analytes are small molecules, such as urea and creatinine, they can be detected using molecularly imprinted polymer (MIP) films. MIP films can act as biomimetic receptors for the detection of analytes (e.g. molecules, proteins or ions) in complex matrices, such as urine. Typically, MIP films are prepared through formation of a polymer network around a template (the target molecule). Template removal via washing results in the formation of cavities, which can be used for target recognition. Advantageously, MIP films are highly selective since cavities replicate the conformation, size and surface chemistry of template molecules. They are also chemically and thermally stable, and fast and inexpensive to produce, making them good alternatives to other bioreceptors, such as antibodies. When target molecules are present in the sample solution, they bind to the cavities within the MIP film. Such an interfacial phenomena can be detected by changes in the impedance.

Accordingly, in one embodiment, there is provided a multi-layered sensor comprising: at least one working electrode layer and at least one reference electrode layer, said working electrode layer and said reference electrode layer being separated by at least one electrically insulating layer; at least two or more polymeric sensing elements disposed on a surface of the working electrode layer; each polymeric sensing element being configured to detect a different target analyte; and means for detecting and measuring changes in an electrical property of the polymeric sensing elements.

In one embodiment, there is provided an in vitro diagnostic kit or a point-of-care kit comprising at least one sensor as described herein.

In another embodiment, the sensor may be integrated with a surface of a fabric that is part of apparel. The apparel may be adapted for casual wear or healthcare use, e.g. adult diapers, baby diapers or an inner lining of pants.

The sensor strips can be integrated with a diaper in two ways: (i) inserted from diaper exterior into the space between the urine absorbent layer and exterior urine-proof layer of the diaper, such that the sensors do not contact wearer's skin; or (ii) attached to the inner surface of the diaper, with a soft paper cover, which prevents direct contact between the sensors and wearer's skin.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1a

FIG. 1a is a schematic illustration showing one possible configuration of the sensor as disclosed herein in a cross-sectional view.

FIG. 1b

FIG. 1b is a schematic illustration showing one possible configuration of the sensor as disclosed herein in a top view.

FIG. 2a

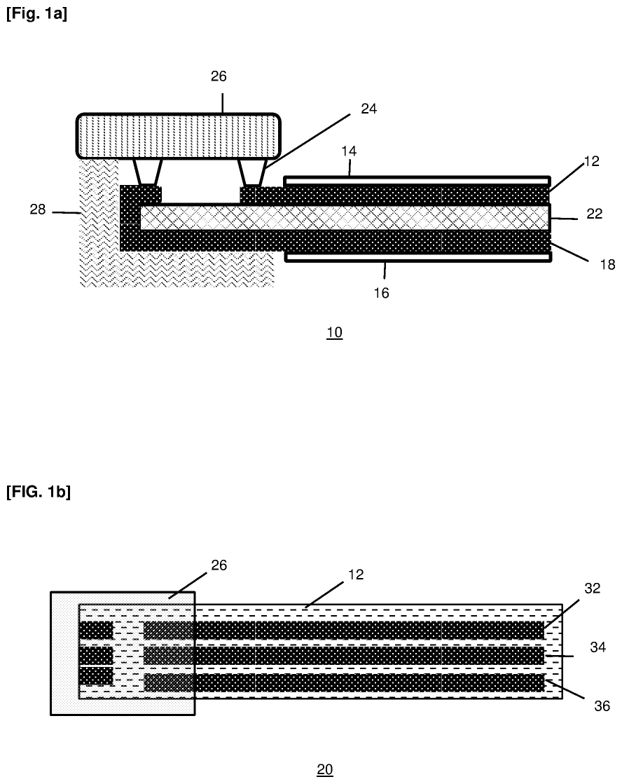

FIG. 1(a) is a graph showing the potentiometric response of a Na⁺ sensor in the detection of Na⁺ in the presence of interference by other ionic species including K⁺, PO₄³⁻, Mg²⁺, Ca²⁺, urea and creatinine. Concentrations of analytes were increased every 100 s.

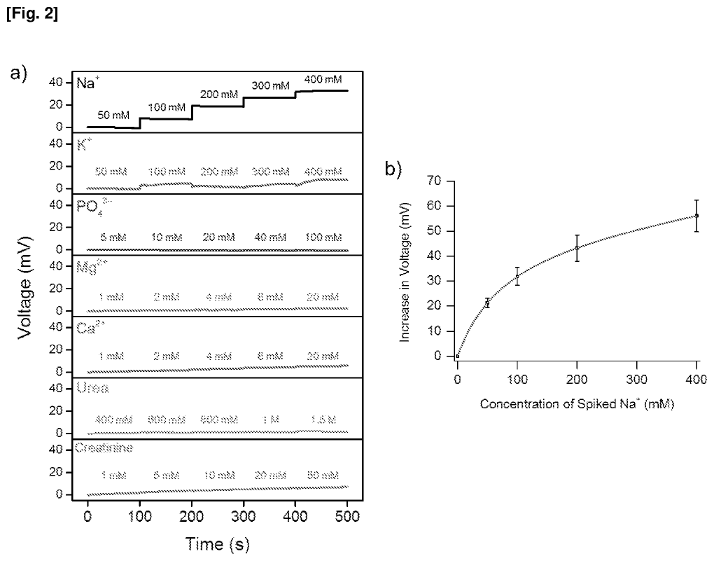

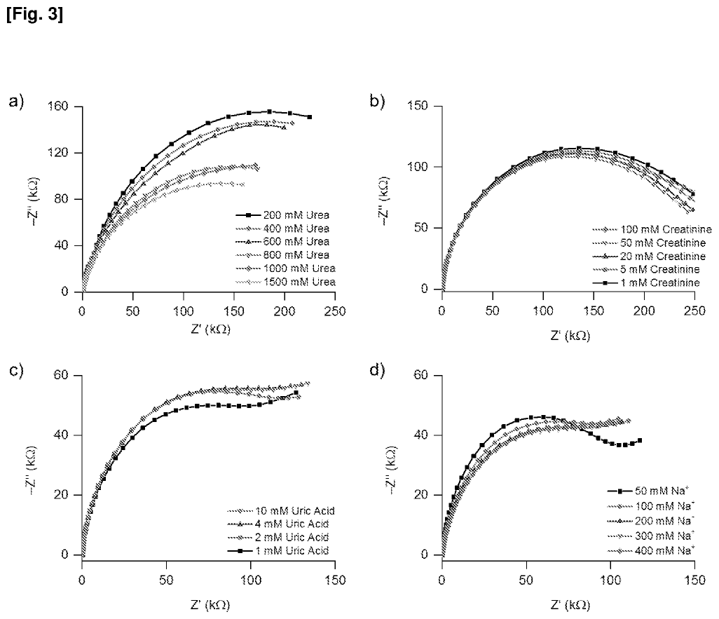

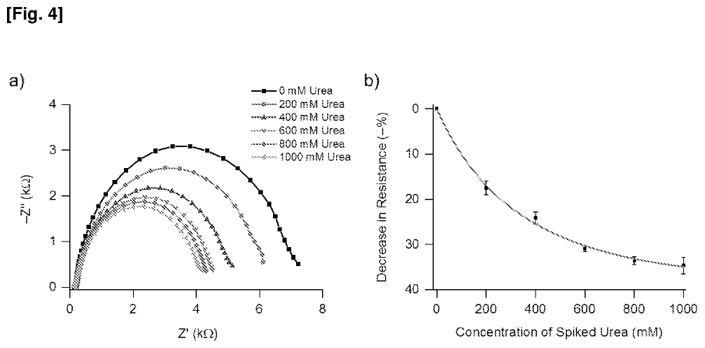

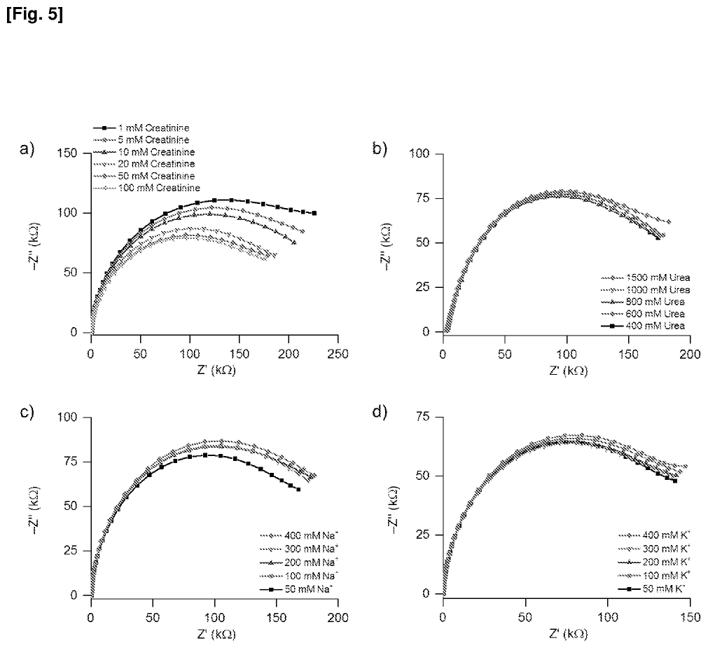

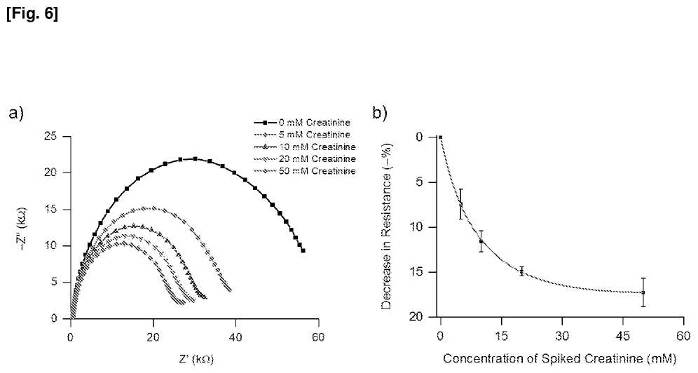

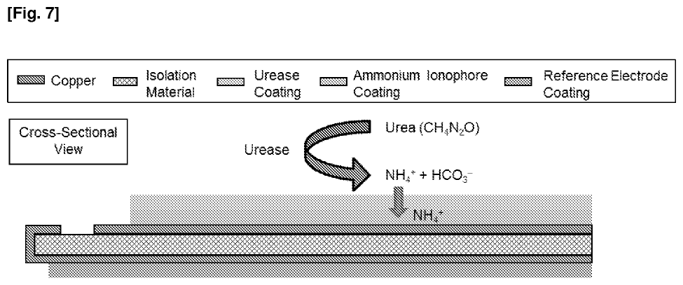

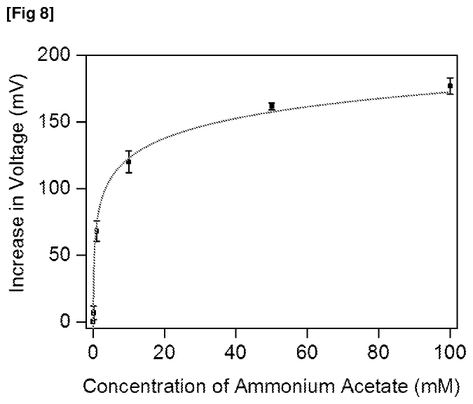

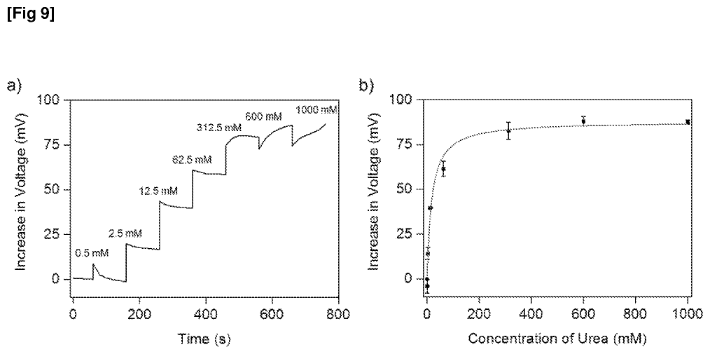

FIG. 2b

FIG. 2(b) is a graph showing the increase in voltage experienced by the Na+ sensor when tested with urine samples that were spiked with increasing concentrations of Na⁺.

FIG. 2a

FIG. 3(a) is a graph showing the electrochemical impedance spectroscopy (EIS) measurements in the presence of (a) urea.

FIG. 3b

FIG. 3(b) is a graph showing the electrochemical impedance spectroscopy (EIS) measurements in the presence of (b) creatinine.

FIG. 3c

FIG. 3(c) is a graph showing the electrochemical impedance spectroscopy (EIS) measurements in the presence of (c) uric acid.

FIG. 3d

FIG. 3(d) is a graph showing the electrochemical impedance spectroscopy (EIS) measurements in the presence of (d) Na⁺.

FIG. 4a

FIG. 4(a) is a graph showing the decrease in (a) impedance obtained by the urea sensor as the concentration of urea spiked into urine increases.

FIG. 4b

FIG. 4(b) is a graph showing the decrease in (b) resistance obtained by the urea sensor as the concentration of urea spiked into urine increases.

FIG. 4a

FIG. 5a is a graph showing EIS measurements in the presence of increasing concentrations of (a) creatinine from 1 to 100 mM.

FIG. 5b

FIG. 5b is a graph showing EIS measurements in the presence increasing concentrations of (b) urea from 400 to 1500 mM.

FIG. 6c

FIG. 5c is a graph showing EIS measurements in the presence of increasing concentrations of (c) Na⁺ from 50 to 400 mM.

FIG. 7d

FIG. 5d is a graph showing EIS measurements in the presence of increasing concentrations of (d) K⁺ from 50 to 400 mM.

FIG. 6a

FIG. 8a. is a graph showing the decrease in (a) impedance obtained by the creatinine sensor as the concentration of creatinine spiked into urine increases.

FIG. 6b

FIG. 9b. is a graph showing the decrease in (b) resistance obtained by the creatinine sensor as the concentration of creatinine spiked into urine increases.

FIG. 10

FIG. 7 is a schematic drawing illustrating the mechanism of voltammetric-based detection of urea by utilizing a dual-layered sensing element comprising a NH4⁺-selective membrane and a urease coating.

FIG. 11.

FIG. 8 is a graph showing an increase in voltage obtained by the urea sensor in the presence of an increasing concentration of ammonium acetate (NH₄CH₃CO₂).

FIG. 9a

FIG. 9a is a graph showing the real-time potentiometric response of the urea sensor in the presence of an increasing concentration of urea, wherein the concentration of urea was increased step-wise every 100 seconds.

FIG. 9b

FIG. 9b is a graph illustrating the increase in voltage obtained with an increasing concentration of urea for a urea sensor.

### DETAILED DESCRIPTION OF DRAWINGS

FIG. 1 shows one configuration of a sensor 10 according to the present disclosure. The modified copper working electrode 12 and reference electrode 18 are separated by an isolation material 22 (e.g. a plastic film), which is electrically insulating, in a three-layered structure. The working electrode 12 may be modified by at least one layer of a polymeric sensing element 14. The reference electrode 18 may also be optionally modified wherein at least one layer of a reference coating 16 is disposed thereon. In a particular embodiment, the reference coating 16 is a Ag/AgCl+ coating.

A portion of the reference electrode 18 wraps around one end of the isolation material 22, so that the connecting points of both the working and reference electrodes are on the same side of the sensor, which can be connected to the connecting pins 24 of the transmitter box 26. The transmitter box 26 measures an electrical signal generated by the sensor strips, and can transmit the data wirelessly to a monitoring computer and software for data analysis (not shown).

The different sensors can be attached onto a single strip of isolation material, and connected to dedicated channels on the transmitter box for multiplexed detection. An embodiment of this is schematically illustrated in FIG. 1b wherein the at least three different sensing elements 32, 34, and 36 are disposed on the copper working electrode 12. Each sensing element is configured to detect a different analyte. Sensing element 32 may be a ion-selective polymer membrane configured to detect sodium ions. Sensing element 34 may be a MIP film configured to detect the presence of urea molecules. Sensing element 36 may be a MIP film configured to detect the presence of creatinine molecules. Each respective sensing element may be connected separately to the transmitted box 26 to provide independent and separate electrical input to the transmitter such that each target analyte can be detected independently.

Another embodiment of the disclosed sensor is illustrated in FIG. 7 wherein a multi-layered polymer sensing element is provided on the electrode. In particular, the schematic illustrates the mechanism of voltammetric-based detection of urea. Similar to the Na⁺ sensor, an ion-selective membrane is required. In this case, ammonium (NH4⁺)-selective membrane is utilized, in addition to a urease coating that is deposited on top of the NH4⁺-selective membrane. In the presence of target urea molecules, urea may be hydrolyzed to NH4⁺+HCO₃⁻. The NH4⁺ generated can then diffuse into the NH4⁺-selective membrane, resulting in a voltage change.

### EXAMPLES

Non-limiting examples of the invention and a comparative example will be further described in greater detail by reference to specific Examples, which should not be construed as in any way limiting the scope of the invention.

### Materials and Methods

### Example 1

### Preparation of Sodium-Selective Polymeric Sensing Element

The following describes the preparation of 1 mL of a Na⁺-selective membrane, which can be scaled up according to the volume required. 241.5 μL of tetrahydrofuran (THF) was mixed with 100 μL of sodium ionophore X (15 g/L in THF), 50 μL of sodium tetrakis[3,5-bis(trifluoromethyl)phenyl]borate (Na-TFPB, 16 g/L in THF), 500 μL of PVC (100 g/L in THF), and 108.5 μL of bis(2-ethylhexyl) sebacate (DOS, neat). The solution was mixed thoroughly, drop-casted onto the surface of the copper tape, and left to dry for at least 1 h at ambient conditions. The modified copper tape can then be used as the working electrode for the detection of Na⁺ in sample solution.

In order to prepare the reference electrode for Na⁺ measurement, another piece of copper tape was coated with Ag/AgCl ink. The coated copper tape was then dried at 120° C. for 1 h.

### Example 2

### Preparation of MIP Films for Urea Sensor

To fabricate the working electrode, a solution of 10 wt % poly(vinyl alcohol-co-ethylene) (10% EVAL) was first prepared in dimethyl sulfoxide (DMSO). Next, template urea molecules were dissolved in the prepared 10% EVAL solution such that urea has a final concentration of 2 wt %. The mixture was then drop-cased on the copper tape, and left to dry overnight at ambient conditions.

For the reference electrode, 1 wt % of template urea molecules were dissolved in 10% EVAL, drop-casted on the copper tape, and left to dry overnight at ambient conditions.

Subsequently, the MIP-coated copper tapes were washed in 50% ethanol solution with mild shaking for 2 h to remove the template urea molecules.

### Example 3

### Preparation of MIP Films for Creatinine Sensor

To prepare the working and reference electrodes, 0.1 and 0.05 wt. % of template creatinine molecules were dissolved in 10% EVAL solution, respectively. The mixtures were then coated on separate copper tapes and left to dry overnight at ambient conditions. The copper tapes were then washed in 50% ethanol solution with mild shaking for 2 h to remove the template creatinine molecules.

### Performance Characterization

### Example 4

### Sodium Sensor

The Na⁺ sensor was prepared by coating the sodium-selective membrane on the copper electrode. FIG. 2a illustrates the open circuit potential (OCP) response of the sodium-selective membrane for the detection of Na⁺ and in the presence of interfering ions and compounds, such as K+, PO₄³⁻, Mg²⁺, Ca²⁺, urea and creatinine.

A distinct increase in voltage was observed with each increase in Na+ concentration. K+ is a common interfering ion due to its similarity in size as compared to Na⁺, but only a slight increase in voltage was observed at 400 mM of K+, which was much higher than the normal daily maximum value of 50-125 mM.

Hence, we do not expect any significant interference from the presence of K+ when trying to detect high concentrations of Na+ during dehydration. In addition, there were relatively insignificant voltage/p.d. changes when electrodes were subjected to other interfering ions and compounds. Therefore, the results show that the disclosed sensor and sensing element is capable of the selective detection of Na+.

To validate the functionality of the Na+-selective sensor, various concentrations of Na+ were spiked into urine collected from a volunteer using NaCl (5 M), and the OCP response in the spiked urine samples was measured.

FIG. 2b illustrates the increase in voltage obtained when the Na+ sensor was tested with urine spiked with an increasing concentration of Na+, demonstrating the feasibility of our sensor in measuring Na+ concentration in physiological urine.

### Example 5

### Urea Sensor

FIG. 3a shows a decrease in impedance as urea concentration increased. In contrast, there were relatively insignificant impedance changes in the presence of interferences, such as creatinine, uric acid and Na+(FIGS. 3b-d, respectively). Therefore, the results show that the disclosed sensor is capable of the specific detection of urea.

Subsequently, we spiked various concentrations of urea into urine collected from a volunteer. FIG. 4a shows a decrease in the impedance obtained when the concentration of spiked urea increased. The resistance was also observed to decrease in the presence of higher urea concentration (FIG. 4b).

### Example 6

### Creatinine Sensor

Likewise, FIG. 5a shows a decrease in impedance as creatinine concentration increased. In contrast, there were relatively insignificant impedance changes in the presence of interferences, such as urea, Na+ and K+(FIGS. 5b-d, respectively). Therefore, specific creatinine detection was achieved.

Various concentrations of creatinine were subsequently spiked into urine collected from a volunteer. FIG. 6a shows a decrease in impedance when the concentration of spiked creatinine in urine was increased. The resistance also decreased in the presence of higher spiked creatinine concentration (FIG. 6b). As the normal physiological creatinine concentration in the urine ranges from 5-20 mM, the disclosed sensor can be used to detect an excess/abnormal amount of creatinine in urine, which can be indicative of renal problems.

### Example 7

Urea Sensor with Multi-Layer Sensing Element

### Preparation of Ammonium-Selective Membrane

The following describes the preparation of 1 mL of the NH4+-selective membrane, which can be scaled up according to the volume required. 397.5 μL of THF was mixed with 120 μL of ammonium ionophore I (15 g/L in THF), 50 μL of Na-TFPB (16 g/L in THF), 366 μL of PVC (100 g/L in THF), and 66.5 μL of DOS (neat). The solution was mixed thoroughly, drop-casted onto the surface of the copper tape, and left to dry for at least 1 h at ambient conditions.

### Preparation of Urease Coating

The volumes described below can be scaled up according to volume required.

Solution A (30 mg/ml Urease in 4% BSA)

| | Volume |
|---|---|
| Component | (μL) |
| 60 mg/ml urease in 50 mM maleic acid-NaOH buffer | 30 |
| pH 6.5 | |
| 10% bovine serum albumin (BSA) | 24 |
| 50 mM maleic acid-NaOH buffer pH 6.5 | 6 |

### Solution B (0.625% Glutaraldehyde)

| Component | Volume (μL) |
|---|---|
| 50% glutaraldehyde (stock solution) | 1 |
| 50 mM maleic acid-NaOH buffer pH | 79 |
| 6.5 | |

Solutions A and B were mixed in a ratio of 12:3 v/v, and drop casted onto the surface of the NH4+-selective membrane. The mixture is left to dry overnight at ambient conditions.

The urease and NH4⁺-selective coatings formed the working electrode of the urea sensor. The reference electrode was prepared as described in Section 2.1.

### Performance Characterization

FIG. 8 below shows the increase in voltage obtained when the urea sensor was subjected to an increasing concentration of ammonium acetate standards.

FIG. 9a shows the real-time potentiometric response of the urea sensor in the presence of an increasing concentration of urea. We observed a distinct increase in voltage with each increase in urea concentration. FIG. 9b illustrates the increase in voltage obtained with an increasing concentration of urea.

### INDUSTRIAL APPLICABILITY

In this work, copper tapes, modified with target-specific polymeric membranes are used as inexpensive material to develop multiplexed sensors that can be integrated with diapers for health screening and monitoring, as well as for the early diagnosis of diseases, such as renal failure. The sensors have been validated for the detection of Na+, urea and creatinine spiked in human urine samples. While the detection of sodium ions, urea and creatinine are expressly exemplified, the sensor may be configured for detecting other types of analytes by making corresponding modifications of the polymeric sensing element (i.e., the MIP film or the ion-selective polymer membrane).

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1.-17. (canceled)

18. A sensor for detecting one or more target analytes, the sensor comprising:
(a) at least one polymeric, sensing element capable of selectively and reversibly binding to a target analyte;
(b) at least one working electrode having the polymeric sensing element disposed thereon;
(c) at least one reference electrode that is electrically communicated with said working electrode; and
(d) means for measuring a change in an electrical property across said working electrode and said reference electrode.

19. The sensor of claim 18, wherein the sensor comprises at least two or more polymeric sensing elements disposed on said working electrode.

20. The sensor of claim 19, wherein each polymeric sensing element is independently configured to detect the same or different target analyte.

21. The sensor of claim 18, wherein each polymeric sensing element is disposed on a surface of the working electrode, said polymeric sensing elements being exposed to an external environment when said sensor is in use.

22. The sensor of claim 18, wherein the electrodes are composed of copper.

23. The sensor of claim 18, wherein each polymeric sensing element is independently selected from an ion-selective polymer membrane or a molecularly imprinted polymer (MIP) film.

24. The sensor of claim 23, wherein the ion-selective polymer membrane comprises an ionophore dispersed within a polymer matrix, said ionophore capable of reversibly forming a complex with said target analyte.

25. The sensor of claim 24, wherein the polymer matrix further comprises at least one additive selected to repel non-target molecules or ions, which are not of the same charge as the target analyte, from the ion-selective polymer membrane.

26. The sensor of claim 23, wherein the ion-selective polymer membrane is prepared from a polymer coating composition comprising at least one polymer, a plasticizer, an ionophore and at least one lipophilic ion additive.

27. The sensor of claim 23, wherein the MIP film is prepared by:
casting a polymer film from a composition comprising a polymer and a target analyte intended for detection by the MIP film;
drying the film; and
removing the target analyte from the dried film to generate cavities thereon, wherein the cavities are specifically adapted to receive the target analyte.

28. The sensor of claim 18, wherein the target analyte is selected from one or more of the group consisting of: Na+, urea, and creatinine.

29. The sensor of claim 18, wherein the working electrode and reference electrode are separated by an electrically insulating layer.

30. The sensor of claim 18, wherein the means for measuring the potential difference comprises at least one transmitter capable of relaying the measured electrical property as electrical signals to an external computer.

31. The sensor of claim 18, wherein said electrical property being measured is selected from voltage, potential difference, impedance or resistance.

32. A multi-layered sensor comprising:
at least one working electrode layer and at least one reference electrode layer, said working electrode layer and said reference electrode layer being separated by at least one electrically insulating layer;
at least two or more polymeric sensing elements disposed on a surface of the working electrode layer; each polymeric sensing element being configured to detect a different analyte; and
means for detecting and measuring changes in an electrical property of the polymeric sensing elements.

33. An in vitro diagnostic kit or a point-of-care kit comprising a sensor for detecting one or more target analytes, the sensor comprising:
(a) at least one polymeric, sensing element capable of selectively and reversibly binding to a target analyte;
(b) at least one working electrode having the polymeric sensing element disposed thereon;
(c) at least one reference electrode that is electrically communicated with said working electrode; and
(d) means for measuring a change in an electrical property across said working electrode and said reference electrode.
